(19) United States
(12) Patent Application Publication
(10) Pub. No.: US 2026/0272962 A1
(43) Pub. Date: Sep. 17, 2026

(54) BORONIC ACID DERIVATIVES AND THERAPEUTIC USES THEREOF

(71) Applicant: Qpex Biopharma, Inc., San Diego, CA (US)

(72) Inventor: Olga LOMOVSKAYA, Mountain View, CA (US)

(21) Appl. No.: 19/668,674

(22) Filed: May 5, 2026

Related U.S. Application Data
(62) Division of application No. 17/619,752, filed on Jun. 17, 2020, now Pat. No. 12,636,297.
(60) Provisional application No. 62/863,752, filed on Jun. 19, 2019.

(51) Int. Cl. A61K 31/69; A61K 31/546; A61P 31/04

(52) U.S. Cl. CPC A61K 31/69; A61K 31/546; A61P 31/04

(57) ABSTRACT

Disclosed herein are antimicrobial compounds compositions, pharmaceutical compositions, the method of use and preparation thereof. Some embodiments relate to boronic acid derivatives and their use as therapeutic agents, for example, β-lactamase inhibitors (BLIs).

### CROSS-REFERENCE TO RELATED APPLICATIONS

This application is a divisional of U.S. application Ser. No. 17/619,752, filed on Dec. 16, 2021, which is a U.S. National Phase Application of International Application No. PCT/US2020/038171, filed on Jun. 17, 2020, designating the United States of America and published in English, which claims the benefit of priority of U.S. Provisional Patent Application No. 62/863,752, filed on Jun. 19, 2019, each of which is incorporated by reference in its entirety.

### BACKGROUND

### Field

The present application relates to the fields of chemistry and medicine. More particularly, the present application relates to boronic acid antimicrobial compounds, compositions, their preparation, and their use as therapeutic agents.

### Description of the Related Art

Antibiotics have been effective tools in the treatment of infectious diseases during the last half-century. From the development of antibiotic therapy to the late 1980s there was almost complete control over bacterial infections in developed countries. However, in response to the pressure of antibiotic usage, multiple resistance mechanisms have become widespread and are threatening the clinical utility of anti-bacterial therapy. The increase in antibiotic resistant strains has been particularly common in major hospitals and care centers. The consequences of the increase in resistant strains include higher morbidity and mortality, longer patient hospitalization, and an increase in treatment costs.

Various bacteria have evolved β-lactam deactivating enzymes, namely, β-lactamases, that counter the efficacy of the various β-lactam antibiotics. β-lactamases can be grouped into 4 classes based on their amino acid sequences, namely, Ambler classes A, B, C, and D. Enzymes in classes A, C, and D include active-site serine β-lactamases, and class B enzymes, which are encountered less frequently, are Zn-dependent. These enzymes catalyze the chemical degradation of β-lactam antibiotics, rendering them inactive. Some β-lactamases can be transferred within and between various bacterial strains and species. The rapid spread of bacterial resistance and the evolution of multi-resistant strains severely limits β-lactam treatment options available.

The increase of class D β-lactamase-expressing bacterium strains such as Acinetobacter baumannii has become an emerging multidrug-resistant threat. A. baumannii strains express A, C, and D class β-lactamases. The class D β-lactamases such as the OXA families are particularly effective at destroying carbapenem type β-lactam antibiotics, e.g., imipenem, the active carbapenems component of Merck's Primaxin® (Montefour, K. et al., Crit. Care Nurse 2008, 28, 15; Perez, F. et al., Expert Rev. Anti Infect. Ther. 2008, 6, 269; Bou, G.; Martinez-Beltran, J., Antimicrob. Agents Chemother. 2000, 40, 428. 2006, 50, 2280; Bou, G. et al., J. Antimicrob. Agents Chemother. 2000, 44, 1556). This has imposed a pressing threat to the effective use of drugs in that category to treat and prevent bacterial infections. Indeed the number of catalogued serine-based β-lactamases has exploded from less than ten in the 1970s to over 300 variants. These issues fostered the development of five “generations” of cephalosporins. When initially released into clinical practice, extended-spectrum cephalosporins resisted hydrolysis by the prevalent class A β-lactamases, TEM-1 and SHV-1. However, the development of resistant strains by the evolution of single amino acid substitutions in TEM-1 and SHV-1 resulted in the emergence of the extended-spectrum β-lactamase (ESBL) phenotype.

New β-lactamases have recently evolved that hydrolyze the carbapenem class of antimicrobials, including imipenem, biapenem, doripenem, meropenem, and ertapenem, as well as other β-lactam antibiotics. These carbapenemases belong to molecular classes A, B, and D. Class A carbapenemases of the KPC-type predominantly in Klebsiella pneumoniae but now also reported in other Enterobacteriaceae, Pseudomonas aeruginosa and Acinetobacter baumannii. The KPC carbapenemase was first described in 1996 in North Carolina, but since then has disseminated widely in the US. It has been particularly problematic in the New York City area, where several reports of spread within major hospitals and patient morbidity have been reported. These enzymes have also been recently reported in France, Greece, Sweden, United Kingdom, and an outbreak in Germany has recently been reported. Treatment of resistant strains with carbapenems can be associated with poor outcomes.

The zinc-dependent class B metallo-β-lactamases are represented mainly by the VIM, IMP, and NDM types. IMP and VIM-producing K. pneumonia were first observed in 1990s in Japan and 2001 in Southern Europe, respectively. IMP-positive strains remain frequent in Japan and have also caused hospital outbreaks in China and Australia. However, dissemination of IMP-producing Enterobacteriaceae in the rest of the word appears to be somewhat limited. VIM-producing enterobacteria can be frequently isolated in Mediterranean countries, reaching epidemic proportions in Greece. Isolation of VIM-producing strains remains low in Northern Europe and in the United States. In stark contrast, a characteristic of NDM-producing K. pneumonia isolates has been their rapid dissemination from their epicenter, the Indian subcontinent, to Western Europe, North America, Australia and Far East. Moreover, NDM genes have spread rapidly to various species other than K. pneumonia.

The plasmid-expressed class D carbapenemases belong to OXA-48 type. OXA-48 producing K. pneumonia was first detected in Turkey, in 2001. The Middle East and North Africa remain the main centers of infection. However, recent isolation of OXA-48-type producing organisms in India, Senegal and Argentina suggest the possibility of a global expansion. Isolation of OXA-48 in bacteria other than K. pneumonia underlines the spreading potential of OXA-48.

Treatment of strains producing any of these carbapenemases with carbapenems can be associated with poor outcomes.

Another mechanism of β-lactamase mediated resistance to carbapenems involves combination of permeability or efflux mechanisms combined with hyper production of beta-lactamases. One example is the loss of a porin combined in hyperproduction of ampC beta-lactamase results in resistance to imipenem in Pseudomonas aeruginosa. Efflux pump over expression combined with hyperproduction of the ampC β-lactamase can also result in resistance to a carbapenem such as meropenem.

Thus, there is a need for improved therapies using β-lactamase inhibitors (BLIs).

### SUMMARY

Some embodiments described herein relate to pharmaceutical compositions comprising a therapeutically effective amount of a compound, or a pharmaceutically acceptable salt thereof, having the structure:

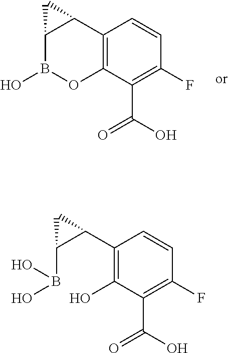 [SMILES: O=C(O)c1c(F)ccc2c1OB(O)[C@@H]1C[C@H]21 ; O=C(O)c1c(F)ccc([C@H]2C[C@H]2B(O)O)c1O]

and a pharmaceutically acceptable excipient; and further comprising an additional medicament, wherein the additional medicament may be ceftolozane or sulbactam.

Other embodiments described herein relate to pharmaceutical compositions comprising a therapeutically effective amount of a compound, or a pharmaceutically acceptable salt thereof, having the structure:

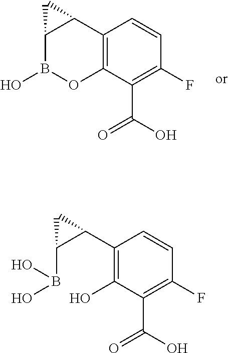 [SMILES: O=C(O)c1c(F)ccc2c1OB(O)[C@@H]1C[C@H]21 ; O=C(O)c1c(F)ccc([C@H]2C[C@H]2B(O)O)c1O]

and a pharmaceutically acceptable excipient; and further comprising an additional medicament, wherein the additional medicament may be cefiderocol.

In some embodiments, the pharmaceutically acceptable salt may be an alkaline metal salt or an ammonium salt. In some embodiments, the sodium salt may be

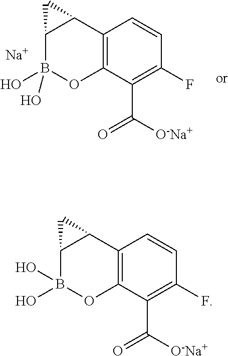

In other embodiments, the sodium salt may be

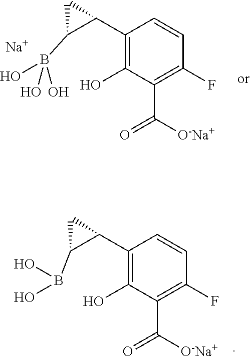

Some embodiments described herein relate to method of treating a bacterial infection, comprising administering to a subject a pharmaceutical composition comprising a compound, or a pharmaceutically acceptable salt thereof, having the structure:

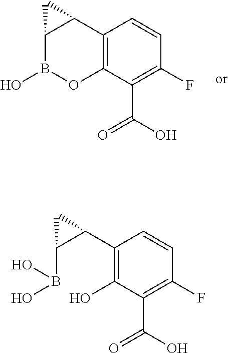 [SMILES: O=C(O)c1c(F)ccc2c1OB(O)[C@@H]1C[C@H]21 ; O=C(O)c1c(F)ccc([C@H]2C[C@H]2B(O)O)c1O]

in combination with ceftolozane or sulbactam.

Some embodiments described herein relate to method of treating a bacterial infection, comprising administering to a subject a pharmaceutical composition comprising a compound, or a pharmaceutically acceptable salt thereof, having the structure:

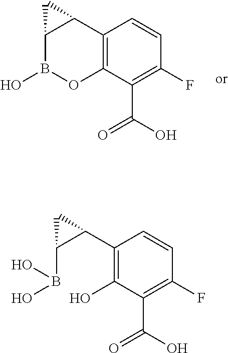 [SMILES: O=C(O)c1c(F)ccc2c1OB(O)[C@@H]1C[C@H]21 ; O=C(O)c1c(F)ccc([C@H]2C[C@H]2B(O)O)c1O]

in combination with cefiderocol.

In some embodiments, the infection may comprise a bacteria selected from the group consisting of Pseudomonas acidovorans, Pseudomonas alcaligenes, Pseudomonas putida, Burkholderia cepacia, Aeromonas hydrophilia, Francisella tularensis, Morganella morganii, Proteus mirabilis, Proteus vulgaris, Providencia alcalifaciens, Providencia rettgeri, Providencia stuartii, Acinetobacter baumannii, Bordetella pertussis, Bordetella para pertussis, Bordetella bronchiseptica, Haemophilus ducreyi, Pasteurella multocida, Pasteurella haemolytica, Branhamella catarrhalis, Borrelia burgdorferi, Kingella, Gardnerella vaginalis, Bacteroides distasonis, Bacteroides 3452A homology group, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus.

In other embodiments, the infection may comprise a bacteria selected from the group consisting of Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, and Bacteroides splanchnicus.

In some specific embodiments, the infection may comprise the bacteria Pseudomonas aeruginosa. In other specific embodiments, the infection may comprise the bacteria Acinetobacter baumannii.

Some embodiments described herein relate to method of treating a bacterial infection, comprising administering to a subject in need thereof a compound or a pharmaceutically acceptable salt thereof, having the structure:

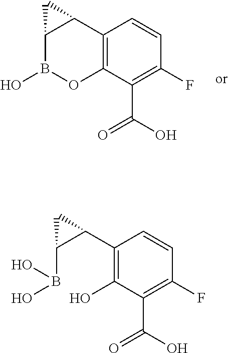 [SMILES: O=C(O)c1c(F)ccc2c1OB(O)[C@@H]1C[C@H]21 ; O=C(O)c1c(F)ccc([C@H]2C[C@H]2B(O)O)c1O]

and an additional medicament, wherein the additional medicament may be ceftolozane or sulbactam.

In some embodiments, the compound and additional medicament may be administered simultaneously. In other embodiments, the compound and additional medicament may be administered sequentially.

In some specific embodiments, the infection may comprise the bacteria Pseudomonas aeruginosa. In other specific embodiments, the infection may comprise the bacteria Acinetobacter baumannii.

### BRIEF DESCRIPTION OF THE DRAWINGS

FIG. 1A shows the minimum inhibitory concentration distribution of Compound 1 combinations against Carbapenem-Resistant Acinetobacter baumannii.

FIG. 1B shows the minimum inhibitory concentration distribution of Compound 1 combinations against Carbapenem-Resistant Acinetobacter baumannii having a defined Carbapenemase.

FIG. 2 shows the minimum inhibitory concentration distribution of meropenem, cefepime and ceftolozane alone and with Compound 1 at 8 μg/mL for Carbapenem-Resistant Enterobacteriaceae. The Figure shows data for Carbapenem-Resistant Enterobacteriaceae lacking metallo-β-lactamases (No MBL; N=264) and having metallo-β-lactamases (MBL; N=145).

### DETAILED DESCRIPTION OF EMBODIMENTS

### Compound 1

Some embodiments provided herein include Compound 1,

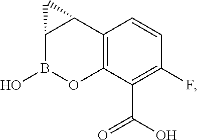 [SMILES: O=C(O)c1c(F)ccc2c1OB(O)[C@@H]1C[C@H]21]

or a pharmaceutically acceptable salt thereof, which contains a boronic acid moiety. Compound 1 can act as an antimicrobial agent and/or as a potentiator of antimicrobial agents.

In some embodiments, the pharmaceutically acceptable salts are selected from alkaline metal salts or ammonium salts. In one embodiment, the pharmaceutically acceptable salts are sodium salts, including disodium salts.

In some embodiments, due to the facile exchange of boron esters, Compound 1 described herein may convert to or exist in equilibrium with alternate forms. Accordingly, in some embodiments, Compound 1 described herein may exist in combination with one or more of these forms. For example, as shown below, Compound 1 disclosed herein may exist in a cyclic boronate monoester with the structure of Compound 1-a depending on the medium. Exemplary equilibrium equation between Compound 1 and Compound 1-a in aqueous medium is demonstrated below:

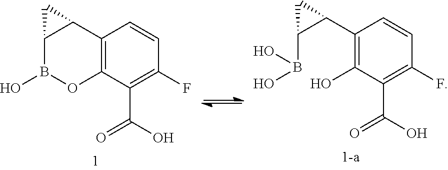 [SMILES: O=C(O)c1c(F)ccc2c1OB(O)[C@@H]1C[C@H]21 ; O=C(O)c1c(F)ccc([C@H]2C[C@H]2B(O)O)c1O]

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. As used in the specification and the appended claims, the singular forms “a,” “an” and “the” include plural referents unless the context clearly dictates otherwise. The use of “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A therapeutic effect relieves, to some extent, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that the structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

Some examples of pharmaceutically acceptable base addition salts of Compound 1 or 1-a disclosed herein have the structure of Compoud 1′ or Compound 1-a′:

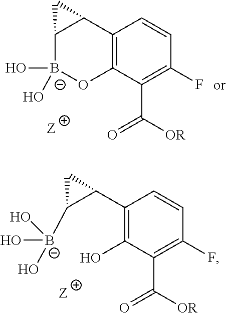

wherein each of Z^{⊕} and R may be independently selected from an alkali metal cation or an ammodium cation (NH₄⁺).

Some examples of pharmaceutically acceptable base addition salts of Compound 1 or 1-a disclosed herein have the structure of Compoud 1″ or Compound 1-a″:

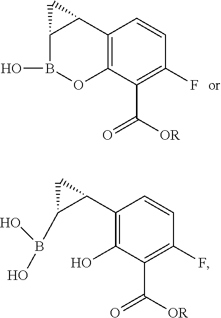

wherein each R may be independently selected from an alkali metal cation or an ammodium cation (NH₄⁺).

In some embodiments, the salt of Compound 1 has the structure

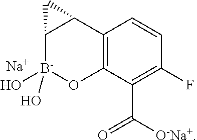

In other embodiments, the salt of Compound 1 has the structure

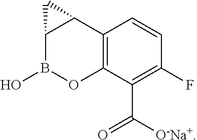

In some embodiments, the salt of Compound 1-a has the structure

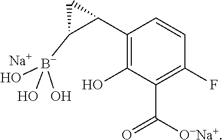

In other embodiments, the salt of Compound 1-a has the structure

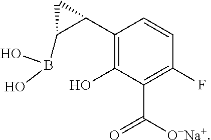

### Methods of Preparation

Compound 1 and 1-a may be synthesized by method described in Example 1, by modification of this method, or using methods disclosed in International Patent Publication Nos. WO 2018/005662 and WO 2018/075084, the entireties of which is incorporated by reference herein. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Administration and Pharmaceutical Compositions

The combination of compound 1 or 1-a and additional medicament are administered at therapeutically effective dosages. In some embodiments, the additional medicament may be ceftolozane or sulbactam. In some embodiments, the additional medicament may be cefiderocol. While human dosage levels have yet to be optimized for the compounds and additional medicaments described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound and additional medicament administered will, of course, be dependent on the subject and disease state being treated, the severity of the affliction, the manner and schedule of administration and the judgment of the prescribing physician.

Administration of the combination of Compound 1 or 1-a and additional medicament disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly.

The combination of Compound 1 or 1-a and additional medicaments useful as described herein can be formulated into pharmaceutical compositions for use in treatment of these conditions. In some embodiments, Compound 1 or 1-a and the additional medicament may be formulated in separate pharmaceutical compositions, whereas in other embodiments, Compound 1 or 1-a and the additional medicament may be formulated in the same pharmaceutical composition. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

Some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound and additional medicament is basically determined by the way the compound and additional medicament is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

In some embodiments, Compound 1 or Compound 1-a and additional medicaments disclosed herein may be formulated into a single unit dosage form suitable for administering to a subject in need thereof. In other embodiments, Compound 1 or Compound 1-a and additional medicaments disclosed herein may be formulated into separate unit dosage forms.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

### Methods of Treatment

Some embodiments of the present invention include methods of treating bacterial infections with Compound 1 or 1a and an additional medicament and compositions comprising Compound 1 or 1a and an additional medicament described herein. Some embodiments include methods of treating bacterial infections with Compound 1 or 1a in combination with an additional medicament selected from the group consisting of sulbactam, ceftolozane, and cefiderocol. Some methods include administering the combination described herein to a subject in need thereof. In some embodiments, a subject can be an animal, e.g., a mammal (including a human). In some embodiments, the bacterial infection comprises a bacteria described herein. As will be appreciated from the foregoing, methods of treating a bacterial infection include methods for preventing bacterial infection in a subject at risk thereof.

In some embodiments, the subject is a human.

Some embodiments include co-administering Compound 1 or 1-a with the additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another such embodiment, administration in combination is accomplished by combining the agents in separate dosage forms. In another embodiment, the agents are administered sequentially. In one embodiment the agents are administered through the same route, such as orally or intravenously. In another embodiment, the agents are administered through different routes, such as one being administered orally and another being administered intravenous (i.v.).

In some embodiments, the additional medicament is selected from ceftolozane and sulbactam. In some embodiments, the additional medicament is cefiderocol.

Some specific embodiments include the combination of Compound 1, or a pharmaceutically acceptable salt thereof, with sulbactam. Other specific embodiments include the combination of Compound 1-a, or a pharmaceutically acceptable salt thereof, with sulbactam. Some specific embodiments include the combination of Compound 1, or a pharmaceutically acceptable salt thereof, with ceftolozane. Other specific embodiments include the combination of Compound 1-a, or a pharmaceutically acceptable salt thereof, with ceftolozane.

Some specific embodiments include the combination of Compound 1, or a pharmaceutically acceptable salt thereof, with cefiderocol. Other specific embodiments include the combination of Compound 1-a, or a pharmaceutically acceptable salt thereof, with cefiderocol.

The combination of Compound 1 or Compound 1-a and additional medicament may further included a second additional medicament. Examples of additional second medicaments include an antibacterial agent, antifungal agent, an antiviral agent, an anti-inflammatory agent and an anti-allergic agent. In some embodiments, the second additional medicaments include a β-lactam. Examples of such β-lactams include Amoxicillin, Ampicillin (e.g., Pivampicillin, Hetacillin, Bacampicillin, Metampicillin, Talampicillin), Epicillin, Carbenicillin (Carindacillin), Ticarcillin, Temocillin, Azlocillin, Piperacillin, Mezlocillin, Mecillinam (Pivmecillinam), Sulbenicillin, Benzylpenicillin (G), Clometocillin, Benzathine benzylpenicillin, Procaine benzylpenicillin, Azidocillin, Penamecillin, Phenoxymethylpenicillin (V), Propicillin, Benzathine phenoxymethylpenicillin, Pheneticillin, Cloxacillin (e.g., Dicloxacillin, Flucloxacillin), Oxacillin, Methicillin, Nafcillin, Faropenem, Biapenem, Doripenem, Ertapenem, Imipenem, Meropenem, Panipenem, Cefazolin, Cefacetrile, Cefadroxil, Cefalexin, Cefaloglycin, Cefalonium, Cefaloridine, Cefalotin, Cefapirin, Cefatrizine, Cefazedone, Cefazaflur, Cefradine, Cefroxadine, Ceftezole, Cefaclor, Cefamandole, Cefminox, Cefonicid, Ceforanide, Cefotiam, Cefprozil, Cefiderocol, Cefbuperazone, Cefuroxime, Cefuzonam, Cefoxitin, Cefotetan, Cefmetazole, Loracarbef, Cefixime, Ceftazidime, Ceftriaxone, Cefcapene, Cefdaloxime, Cefdinir, Cefditoren, Cefetamet, Cefmenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, Flomoxef, Latamoxef, Cefepime, Cefozopran, Cefpirome, Cefquinome, Ceftobiprole, Ceftaroline, Ceftiofur, Cefquinome, Cefovecin, Aztreonam, Tigemonam and Carumonam.

### Indications

The compounds and compositions comprising the compounds described herein can be used to treat bacterial infections. Bacterial infections that can be treated with the compounds, compositions and methods described herein can comprise a wide spectrum of bacteria. Example organisms include gram-positive bacteria, gram-negative bacteria, aerobic and anaerobic bacteria, such as Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella and other organisms.

More examples of bacterial infections include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

Some preferred embodiments are directed to treating infections comprising the bacteria Acinetobacter baumannii. Other preferred embodiments are directed to treating infections comprising the bacteria Pseudomonas aeruginosa.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### EXAMPLES

### General Procedures

Materials used in preparing the cyclic boronic acid ester derivative described herein may be made by known methods or are commercially available. It will be apparent to the skilled artisan that methods for preparing precursors and functionality related to the compounds claimed herein are generally described in the literature including, for example, procedures described in U.S. Pat. No. 7,271,186, International Patent Publication No. WO 2009/064414, and International Patent Publication No. WO 2018/005662, each of which is incorporated by reference in its entirety. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example is provided for the guidance of the reader, and represent preferred methods for making the compound exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

### Example 1

### Disodium Salt of 5-Fluoro-2-hydroxy-1a,7b-dihydro-1H-cyclopropa[c][1,2]benzoxaborinine-4-carboxylic acid (Compound 1′)

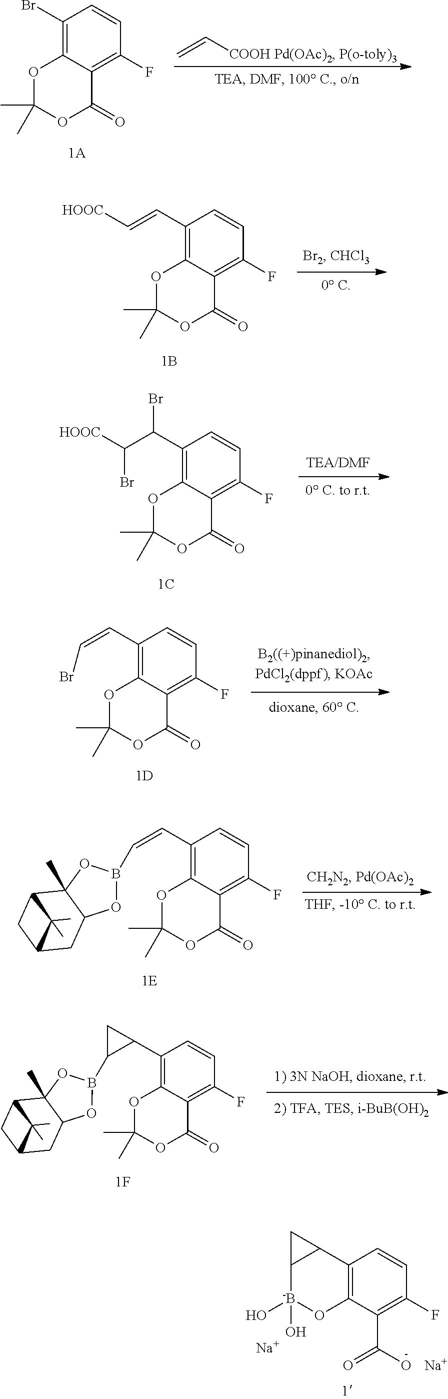

### Step 1: Synthesis of Compound 1A

Compound 1A was prepared from Boc-t-Butyl ester intermediate (previously disclosed in WO 2015/179308) by TFA deprotection followed by isopropylidene protection as described in step 2 of Example 1.

### Step 2: Synthesis of Compound 1B

To the solution of compound 1A (16.0 g, 58 mmol, 1.0 eq) in DMF (50 mL) was added acrylic acid (6.0 mL, 87 mmol, 1.5 eq), TEA (24 mL, 175 mmol, 3 eq), Pd(OAc)₂ (651 mg, 2.9 mmol, 0.05 eq) and tri(o-tolyl)phosphine (1.77 g, 5.8 mmol, 0.1 eq). The reaction mixture was flushed with nitrogen and stirred at 100° C. for 14 hours. The reaction mixture was concentrated to dryness and the solid was washed with 0.2N HCl and DCM to give a yellow solid. The solid was re-crystallized in EtOAc and hexanes to give compound 1B (8.2 g, 53%) as an off-white solid. ¹H NMR (CD₃OD, 400 MHz): δ8.01 (dd, 1H), 7.78 (d, J=16.4 Hz, 1H), 7.00 (dd, 1H), 6.57 (d, J=16.0 Hz, 1H), 1.80 (s, 6H).

### Step 3: Synthesis of Compound 1C

To the suspension of compound 1B (8.2 g, 30.8 mmol, 1.0 eq) in chloroform (300 mL) was added bromine liquid (1.8 mL, 35.4 mmol, 1.15 eq) dropwise in 5 minutes at 0° C. The reaction solution was stirred at 0° C. for 2 hours before it was concentrated under reduced pressure. The obtained yellow solid is crude compound 1C (14.7 g), which was used directly for next step without purification.

### Step 4: Synthesis of Compound 1D

To the solution of compound 1C (14.7 g, 30.8 mmol, 1.0 eq) in DMF (35 mL) was added triethylamine (8.6 mL, 61.6 mmol, 2.0 eq) dropwise in 2 minutes at 0° C. The resulting reaction mixture was slowly warmed up to rt and stirred for 8 hours. The reaction mixture was diluted with EtOAc and washed with 0.1N HCl and water. After dried over Na₂SO₄, the organic layer was concentrated and chromatography (hexanes/EtOAc=3/1 to 1/1) to give compound 4D (5.5 g) as an off-white solid. ¹H NMR (CDCl₃, 400 MHz): δ8.20 (dd, 1H), 7.08 (d, J=8.0 Hz, 1H), 6.88 (t, J=8.2 Hz, 1H), 6.55 (d, J=8.0 Hz, 1H), 1.75 (s, 6H).

### Step 5: Synthesis of Compound 1E

The mixture of compound 1D (700 mg, 2.3 mmol, 1.0 eq), bis((+)pinanediolato)diboron (1.24 g, 3.5 mmol, 1.5 eq), PdCl₂ (dppf) (188 mg, 0.23 mmol, 0.1 eq) and KOAc (450 mg, 4.6 mmol, 2.0 eq) in dioxane (15 mL) was stirred at 60° C. for 2 hours under nitrogen atmosphere. The reaction mixture was diluted with EtOAc and washed with 0.1N HCl and water. After dried over Na₂SO₄, the organic layer was concentrated and purified by column chromatography (hexanes/EtOAc=3/1 to 1/1) to give compound 1E (240 mg, 26%) as a yellow solid. ESI-MS: [M+H]⁺: 401.

### Step 6: Synthesis of Compound 1F

To the solution of compound 1E (240 mg, 0.6 mmol, 1.0 eq) and Pd(OAc)₂ (6.8 mg, 0.03 mmol, 0.05 eq) in THF (3 mL) was slowly added diazomethane (6 mL, freshly made, about 0.2 to 0.3 M in ether) at −10° C. in 15 minutes. The solution was slowly warmed up to rt and stirred for 2 hours before it was concentrated to dryness. The obtained residue was and purified by column chromatography (hexanes/EtOAc=3/1 to 1/1) to give compound 1F (240 mg, 99%) as yellow oil. ESI-MS: [M+H]⁺: 415.

### Step 7: Synthesis of Compound 1′

The mixture of compound 1F (140 mg, 0.34 mmol, 1.0 eq) in dioxane (1.5 mL) and 3N NaOH (1.5 mL) was stirred at rt for 1 hour, LCMS indicating the disappearance of starting material. The reaction mixture was cooled to 0° C. and TES (250 mg), TFA (5 mL) and i-BuB(OH)₂ (100 mg) was added in sequence. The resulting yellow clear solution was stirred at rt for 2 hours before it was concentrated to dryness. The residue was dissolved in water/MeCN and purified by prep-HPLC (C18, acetonitrile and water as mobile phases, 0.1% TFA). The obtained solid (28 mg) was dissolved in MeCN/water and adjusted to pH=9.5 with 1N NaOH (0.27 mL). After lyophilization, the crude sodium salt of Compound 1 was dissolved in 1.0 mL water and acetone (8.0 mL) was added dropwise. The resulting suspension was stirred at rt for 3 hours. The mixture was filtered and the solid was washed with 10% water in acetone twice to give Compound 1′ (26 mg) as an off-white solid. ¹H NMR (D₂O, 300 MHz): δ6.87 (t, J=7.2 Hz, 1H), 6.25 (d, J=8.4 Hz, 1H), 1.65-1.56 (m, 1H), 0.67-0.57 (m, 1H), 0.14-0.03 (m, 2H). F NMR (D₂O, 300 MHz): δ−124.9. ESI-MS: [M−H₂O+H]⁺: 205.

### Example 2

Embodiments of the present application are disclosed in further detail in the following example, which is not in any way intended to limit the scope of the present disclosure.

In this example the in vitro activity of Compound 1 was evaluated in combination with several β-lactams including sulbactam, meropenem, and ceftolozane against clinical isolates of carbapenem-resistant (CR) Acinetobacter baumannii (AB) and Pseudomonas aeruginosa (PA).

A total of 300 of carbapenem-resistant Acinetobacter baumannii (CRAB (including a subset of 100 CRAB strains containing defined carbapenemases and 1000 PA clinical isolates were tested by the reference broth microdilution method against β-lactams alone and combined with Compound 1 (4 μg/mL and 8 μg/mL). 500 PA isolates were selected to represent the normal distribution of meropenem (MER), ceftazidime-avibactam (CAZ-AVI), and ceftolozane-tazobactam (TOL-TAZ) resistance according to 2017 surveillance data (representative panel). Additionally, 652 PA isolates that were either non-susceptible (NS) to MER (Minimum Inhibitory Concentration (MIC), >2 μg/mL) or to TOL-TAZ (MIC, >4 μg/mL), or resistant (R) to CAZ-AVI (MIC, >8 μg/mL) (challenge panel) were also tested.

The activity data for Compound 1 in combination with sulbactam against representative AB strains are shown in Table 1. The data shows that the combination of Compound 1 with sulbactam improves the minimum inhibitory concentration of sulbactam alone against a variety of AB strains.

TABLE 1
Activity (Minimum Inhibitory Concentration (μg/mL))
of Compound 1 in Combination with Sulbactam
(SUL) against Representative Strains

| | | | SUL + | SUL + |
|---|---|---|---|---|
| | | | Compound 1 | Compound 1 |
| Strain | Carbapenemase | SUL | at 4 μg/mL | at 8 μg/mL |
| AB1291 | KPC-2 | >64 | 0.25 | 0.05 |
| AB1358 | NDM-1 | >64 | 16 | 4 |
| AB1144 | NDM-1 | >64 | 4 | 0.25 |
| AB1359 | NDM-1 | >64 | 2 | ≤0.06 |
| AB1227 | OXA-23 | 64 | 8 | 8 |
| AB1244 | OXA-23 | 64 | 32 | 32 |
| AB1307 | OXA-23 | 32 | 4 | 2 |
| AB1333 | OXA-239 | 16 | 2 | 2 |
| AB1192 | OXA-24, | 32 | 4 | 4 |
| | OXA-72 | | | |
| AB1223 | OXA-72 | 4 | 0.25 | ≤0.06 |

The minimum inhibitory concentration (MIC) distribution of various combinations of Compound 1 for the representative panel of PA is provided in Table 2. The combination of Compound 1 and ceftolozane has excellent potency against the representative panel of P. aeruginosa that reflects current MIC distributions. The combination of Compound 1 and ceftolozane is more potent than the combination of Compound 1 and meropenem, and more potent than the combinations of ceftolozane-tazobactam (TOL-TAZ) and ceftazidime-avibactam (CAZ-AVI) against this panel of isolates.

TABLE 2
Minimum Inhibitory Concentration Distributions of Compound
1 Combinations for the Representative Panel of PA (N = 500)

| | | MER + | MER + | | | MER + | TOL + | |
|---|---|---|---|---|---|---|---|---|
| MIC | | Compound 1 | Compound 1 | | | Compound 1 | Compound 1 | |
| (μg/mL) | MER | at 4 μg/mL | at 8 μg/mL | TOL | TOL + TAZ | at 4 μg/mL | at 8 μg/mL | CAZ + AVI |
| ≤0.06 | 4.8% | 9.0% | 10.8% | 0.2% | 0.0% | 0.6% | 2.4% | 0.2% |
| 0.125 | 14.2% | 26.0% | 29.0% | 0.2% | 0.2% | 2.6% | 3.4% | 0.2% |
| 0.25 | 33.0% | 47.8% | 50.8% | 2.8% | 3.6% | 8.0% | 13.2% | 0.8% |
| 0.5 | 51.0% | 61.0% | 62.6% | 56.2% | 55.8% | 66.0% | 69.6% | 4.0% |
| 1 | 66.2% | 70.6% | 71.6% | 77.4% | 80.6% | 89.2% | 91.2% | 28.4% |
| 2 | 73.2% | 82.0% | 82.8% | 85.2% | 88.6% | 93.8% | 96.4% | 66.8% |
| 4 | 79.8% | 85.8% | 86.4% | 90.4% | 91.8% | 95.6% | 97.6% | 82.4% |
| 8 | 84.8% | 91.0% | 91.6% | 92.2% | 93.8% | 97.4% | 97.8% | 92.2% |
| 16 | 90.4% | 95.8% | 96.2% | 93.0% | 93.8% | 98.2% | 98.2% | 94.8% |
| 32 | 95.4% | 98.0% | 98.2% | 93.2% | 99.8% | 98.4% | 98.4% | 97.0% |
| >32 | 100.0% | 100.0% | 100.0% | 100.0% | 100.0% | 100.0% | 100.0% | 100.0% |

MER, meropenem;
TOL, ceftolozane;
SUL, sulbactam;
CAZ, ceftazidime;
AVI, avibactam,
TAZ, tazobactam.
AVI and TAZ at 4 μg/mL

The MIC distribution of combinations of Compound 1 with various antibiotics for the challenge panel of Pseudomonas aeruginosa is provided in Table 3. The data shows that the combination of Compound 1 with ceftolozane have increased potency against the challenge panel of PA. The combination of Compound 1 with ceftolozane is more potent than the combination of Compound 1 with meropenem, and more potent than the combinations of ceftolozane-tazobactam (TOL-TAZ) and ceftazidime-avibactam (CAZ-AVI) against the challenge panel of PA enriched in ceftazidime-avibactam resistant and meropenem, caftolozane-tazobactam non-susceptible isolates.

TABLE 3
MIC Distributions of Compound 1 (C1) Combinations
for the Representative Panel of PA (N = 500)

| | MER NS (MER >2 μg/mL), N = 612 | | | | | CAZ-AVI R (CAZ-AVI >8 μg/mL) | | |
|---|---|---|---|---|---|---|---|---|
| MIC | | MER + | TOL + | TOL + | CAZ + | | MER + | TOL + |
| (μg/mL) | MER | C1 | TAZ | C1 | AVI | MER | C1 | TAZ |
| ≥0.06 | 0.0% | 1.3% | 12.4% | 0.5% | 0.0% | .3% | 1.6% | 26.0% |
| 0.125 | 0.0% | 1.6% | 12.4% | 0.7% | 0.0% | .3% | 3.2% | 26.0% |
| 0.25 | .0% | 2.1% | 12.6% | 2.0% | 0.0% | .6% | 5.2% | 26.0% |
| 0.5 | .0% | 4.6% | 20.6% | 18.5% | 0.2% | .2% | 8.8% | 26.3% |
| 1 | .0% | 7.0% | 39.1% | 52.3% | 2.8% | .5% | 12.0% | 26.9% |
| 2 | .0% | 26.5% | 48.2% | 72.1% | 14.4% | .8% | 19.8% | 28.9% |
| 4 | .5% | 41.3% | 55.4% | 83.8% | 36.1% | 1.0% | 33.8% | 36.4% |
| 8 | 7.5% | 63.9% | 63.1% | 87.4% | 53.6% | 8.2% | 6.2% | 4.8% |
| 16 | 6.7% | 83.2% | 8.3% | 89.7% | 66.5% | 8.9% | 3.1% | 8.7% |
| 32 | 0.4% | 92.2% | 5.1% | 91.0% | 82.0% | 3.2% | 5.7% | 0.3% |
| >32 | 100.0% | 100.0% | 00.0% | 100.0% | 100.0% | 100.0% | 100.0% | 100.0% |

| | CAZ-AVI R (CAZ-AVI >8 μg/mL) | | TOL-TAZ NS (TOL-TAZ >4 μg/mL) | | | | |
|---|---|---|---|---|---|---|---|
| MIC | TOL + | CAZ + | | MER + | TOL + | TOL + | CAZ + |
| (μg/mL) | C1 | AVI | MER | C1 | TAZ | C1 | AVI |
| ≥0.06 | 0.3% | 0.0% | 0.3% | 3.0% | 0.0% | 1.3% | 0.0% |
| 0.125 | 0.6% | 0.0% | 1.3% | 5.0% | 0.0% | 1.3% | 0.0% |
| 0.25 | 0.6% | 0.0% | 2.7% | 8.7% | 0.0% | 2.3% | 0.3% |
| 0.5 | 3.2% | 0.0% | 3.7% | 13.4% | 0.0% | 9.4% | 0.3% |
| 1 | 21.8% | 0.0% | 6.0% | 16.7% | 0.0% | 33.1% | 1.0% |
| 2 | 45.5% | 0.0% | 8.7% | 35.5% | 0.0% | 55.9% | 6.0% |
| 4 | 67.2% | 0.0% | 13.7% | 49.8% | 0.0% | 72.9% | 21.7% |
| 8 | 74.7% | 0.0% | 22.1% | 70.6% | 20.4% | 78.6% | 34.4% |
| 16 | 79.2% | 29.9% | 32.8% | 83.9% | 32.1% | 82.6% | 47.2% |
| 32 | 82.5% | 62.7% | 58.2% | 90.0% | 89.3% | 85.3% | 70.6% |
| >32 | 100.0% | 100.0% | 100.0% | 100.0% | 100.0% | 100.0% | 100.0% |

Compound 1 at 8 μg/mL;
AVI and TAZ at 4 μg/mL

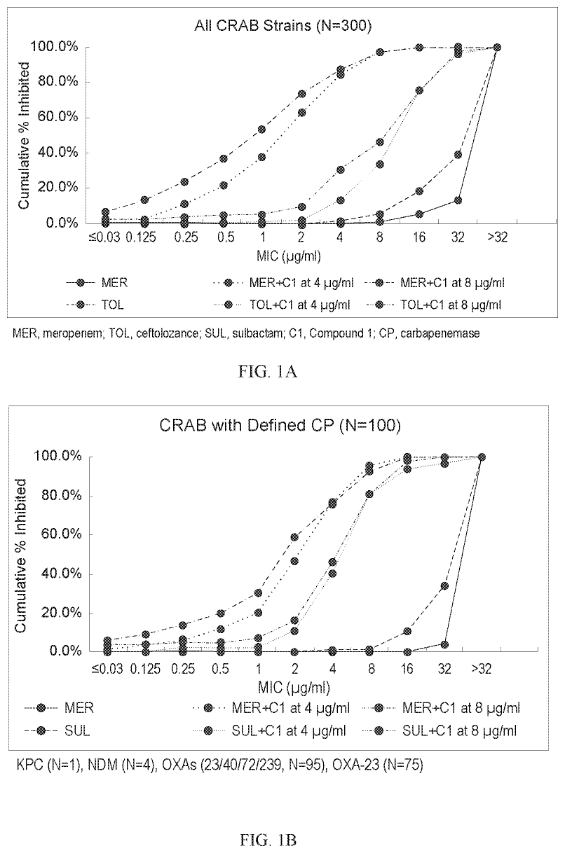

The activity of Compound 1 combinations with meropenem (MER), ceftolozane (TOL), and sulbactam (SUL) is shown in Table 4. Combinations of Compound 1 with various β-lactam antibiotics displayed potent activity against CRAB and PA isolates as shown in Table 4 and FIGS. 1A and 1B. The combination of Compound 1 with TOL exhibited activity against 300 CRAB, including 100 CRAB with defined CPs as well as against representative and challenge PA strains and restored susceptibility(S) to TOL. TOL-Compound 1 was also more potent compared to TOL-TAZ and CAZ-AVI against TOL-TAZ- and CAZ-AVI-resistant isolates.

TABLE 4
Activity of Combinations of Compound 1 with various β-lactam antibiotics

| | MIC₅₀/MIC₉₀ (μg/mL) (% susceptible, [S]) | | | | | |
|---|---|---|---|---|---|---|
| | | MER + | | TOL + | | SUL + |
| Organisms (no.) | MER | Compound 1 | TOL | Compound 1 | SUL | Compound 1 |
| CRAB (300) | >32/>32 | 2/8 | >32/>32 | 16/32 | ND | ND |
| | (1.0) | (97.0) | (1.3) | (13.7) | | |
| AB CP (100) | >32/>32 | 4/8 | >32/>32 | 16/32 | >32/>32 | 8/16 |
| | (0.0) | (96.0) | (0.0) | (12) | | |
| | | MER + | | TOL + | TOL + | |
| | MER | Compound 1 | TOL | Compound 1 | TAZ | CAZ-AVI |
| PA (Representative, 500) | 0.5/16 | 0.5/8 | 0.5/4 | 0.5/2 | 0.5/4 | 2/8 |
| | (84.8) | (85.8) | (90.4) | (95.6) | (91.8) | (92.2) |
| PA (Challenge, 652)) | 16/>32 | 8/32 | 16/>32 | 2/32 | 4/32 | 8/>32 |
| | (31.9) | (60.7) | (44.0) | (75) | (54) | (52.8) |
| PA (MER MIC >2) (612) | 32/>32 | 8/>32 | 4/>32 | 2/>32 | 4/>32 | 8/>32 |
| | (27.5) | (58.2) | (56.2) | (74.5) | (55.4) | (53.6) |
| PA (TOL-TAZ MIC >4) (39) | 32/>32 | 8/>32 | >32/>32 | 8/>32 | >32/>32 | 32/>32 |
| | (21.1) | (64.2) | (3.3) | (58.2) | (0.0) | (34.4) |
| PA (CAZ-AVI MIC >8) (37) | 32/>32 | 16/>32 | >32/>32 | 8/>32 | 32/32 | >32/>32 |
| | (18.9) | (49.0) | (18.2) | (49.0) | (36.4) | (0.0) |

MER, meropenem;
TOL, ceftolozane;
SUL, sulbactam;
CAZ, ceftazidime;
AVI, avibactam.
Compound 1, AVI, and TAZ were tested at fixed 4 μg/mL.
Breakpoints:
MER ≤8 μg/mL;
TOL ≤4 μg/mL:
CAZ ≤8 μg/mL.

In addition, 509 Carbapenem-Resistant Enterobacteriaceae were tested with meropenem, cefepime, and ceftolozane alone and in combination with Compound 1 at 8 μg/mL. Furthermore, 262 strains of P. aeruginosa with a meropenem MIC>2 μg/mL were tested with meropenem, ceftolozane, cefepime and piperacillin alone and in combination with Compound 1 at 8 μg/mL.

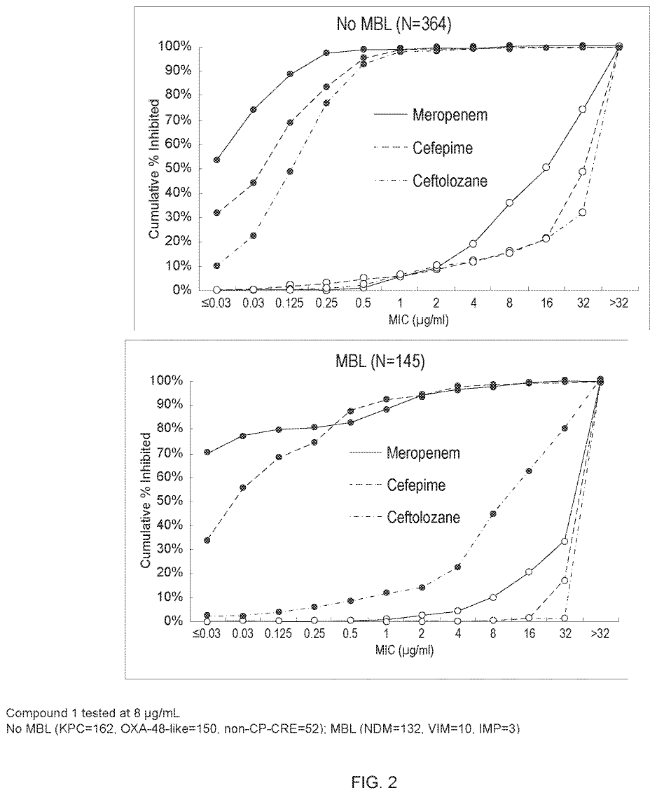

The MIC distribution of combinations of Compound 1 with meropenem, cefepime and cetolozane alone, or with Compound 1 at 8 μg/mL for Carbapenem-Resistant Enterobacteriaceae were tested. Compound 1 significantly increased potency of all tested antibiotics and restores the petency of meropenem, cefepime, and ceftolozane against Carbapenem-Resistant Enterobacteriaceae (FIG. 2).

Compound 1 also increased the proportion of susceptible P. aeruginosa. 262 strains of P. aeruginosa having a meropenem MIC greater than 2 μg/mL were tested with meropenem, ceftolozane, cefepime and piperacillin alone and Compound 1 at 8 μg/mL. Table 5 shows changes in % of susceptible organisms at breakpoints. Compound 1 increased the proportion of susceptible organisms for all tested agents.

TABLE 5
Susceptibility Testing of P. aeruginosa with meropenem (MER), ceftolozane
(TOL), cefepime (FEP) and piperacillin (PP) alone and Compound 1 at 8 μg/mL

| | | MER + | | TOL + | | FEP + | PIP + | PIP + |
|---|---|---|---|---|---|---|---|---|
| | MER | Compound 1 | TOL + TAZ | Compound 1 | FEP | Compound 1 | TAZ | Compound 1 |
| All strains | 42 | 66 | 49 | 77 | 20 | 65 | 17 | 76 |
| (N = 262) | | | | | | | | |
| No MBL | 51 | 75 | 61 | 92 | 24 | 76 | 19 | 80 |
| (N = 206) | | | | | | | | |
| MBL | 7 | 30 | 5 | 23 | 4 | 23 | 7 | 61 |
| (N = 56) | | | | | | | | |

Breakpoints:
≤8 μg/mL for meropenem and cefepime;
≤4 μg/mL for ceftolozane;
≤16 μg/mL for piperacillin;
MBL (metallo-β-lactamase)

### Example 3

Embodiments of the present application are disclosed in further detail in the following example, which is not in any way intended to limit the scope of the present disclosure.

In this example, the antiobiotics meropenem, cefepime, ceftolozane and cefiderocol, were tested in combination with Compound 1 at 4 μg/ml and 8 μg/ml against a panel of 510 clinical isolates representing various types of carbapenem resistant Enterobacteriaceae (CRE). Cefiderocol was tested at standard conditions, in iron proficient media. MIC90 of all tested antibiotics against this panel were in resistant range and Compound 1 shifted MIC90 of all antibiotics (except ceftolozane tested against metallo beta-lactamases, MBL) into susceptible range. The results are presented in Table 6 and show that Compound 1 significantly enhances the potency of these antibiotics against carbapenem resistant Enterobacteriaceae

TABLE 6
Activity of Compound 1 in combination with meropenem (MER), cefepime (FEP), ceftolozane
(TOL) and cefiderocol (DER) against various types of CRE (N = 510)

| | | | | | | | | | | | |
|---|---|---|---|---|---|---|---|---|---|---|---|
| | MER | MER | | FEP | FEP | | TOL | TOL | | DER | DER |
| | w/QPX | w/QPX | | w/QPX | w/QPX | | w/QPX | w/QPX | | w/QPX | w/QPX |
| | at 4 | at 8 | | at 4 | at 8 | | at 4 | at 8 | | at 4 | at 8 |
| MER | μg/ml | μg/ml | FEP | μg/ml | μg/ml | TOL | μg/ml | μg/ml | DER | μg/ml | μg/ml |

KPC, N = 168

| | | | | | | | | | | | | |
|---|---|---|---|---|---|---|---|---|---|---|---|---|
| MIC50 | 32 | <=0.06 | <=0.06 | 32 | 0.125 | 0.06 | >32 | 0.25 | 0.125 | 2 | 0.125 | 0.06 |
| MIC90 | >32 | 0.25 | 0.125 | >32 | 0.5 | 0.25 | >32 | 1 | 0.5 | 32 | 2 | 1 |

OXA, N = 152

| | | | | | | | | | | | | |
|---|---|---|---|---|---|---|---|---|---|---|---|---|
| MIC50 | 32 | 0.06 | <=0.06 | >32 | 0.25 | 0.125 | >32 | 0.5 | 0.25 | 1 | 0.125 | 0.06 |
| MIC90 | >32 | 0.125 | 0.125 | >32 | 0.5 | 0.5 | >32 | 1 | 0.5 | 8 | 0.5 | 0.5 |

non-carbapenemase producing CRE, N = 45

| | | | | | | | | | | | | |
|---|---|---|---|---|---|---|---|---|---|---|---|---|
| MIC50 | 8 | 0.25 | 0.125 | >32 | 0.5 | 0.125 | >32 | 0.5 | 0.25 | 2 | 0.5 | 0.25 |
| MIC90 | 16 | 2 | 0.5 | >32 | 2 | 1 | >32 | 8 | 1 | 32 | 4 | 2 |

MBL, N = 145

| | | | | | | | | | | | | |
|---|---|---|---|---|---|---|---|---|---|---|---|---|
| MIC50 | >32 | 0.06 | <=0.06 | >32 | 0.25 | 0.06 | >32 | >32 | 16 | 8 | 0.125 | 0.06 |
| MIC90 | >32 | 8 | 2 | >32 | 8 | 1 | >32 | >32 | >32 | >32 | 1 | 0.5 |

MER = meropenem;
FEP = cefepime;
TOL - ceftolozane;
DER = cefiderocol;
QPX = Compound 1

### Example 4

Embodiments of the present application are disclosed in further detail in the following example, which is not in any way intended to limit the scope of the present disclosure.

In another experiment, various antibiotics, meropenem, cefepime, ceftolozane and cefiderocol, were tested in combination with Compound 1 at 4 μg/ml and 8 μg/ml against the panel of 503 clinical isolates of carbapenem resistant Acinetobacter baumannii. Compound 1 significantly enhances the potency of meropenem and cefiderocol against this panel of highly resistant strains shifting MIC90 of both antibiotics into susceptible level. Cefiderocol was tested at standard conditions, in iron proficient media. The results are presented in Table 7.

TABLE 7
Activity of Compound 1 in combination with various antibiotics against
carbapenem resistant Acinetobacter baumannii (N = 503)

| | | MER- | MER- | | FEP- | FEP- | | TOL- | TOL- | | DER- | DER- |
|---|---|---|---|---|---|---|---|---|---|---|---|---|
| | | QPX | QPX | | QPX | QPX | | QPX | QPX | | QPX | QPX |
| | | at 4 | at 8 | | at 4 | at 8 | | at 4 | at 8 | | at 4 | at 8 |
| | MER | μg/ml | μg/ml | FEP | μg/ml | μg/ml | TOL | μg/ml | μg/ml | DER | μg/ml | μg/ml |
| MIC₅₀ | >32 | 1 | 0.5 | >32 | 16 | 16 | >32 | 16 | 8 | 1 | 0.5 | 0.5 |
| MIC₉₀ | >32 | 8 | 4 | >32 | >32 | 32 | >32 | 32 | 32 | 32 | 2 | 2 |

Although the present disclosure has been described with reference to embodiments and examples, it should be understood that numerous and various modifications can be made without departing from the spirit of the present disclosure. Accordingly, the present disclosure is limited only by the following claims.

All references cited herein, including patents, patent applications, papers, text books, and the like, and the references cited herein, to the extent that they are not already, are hereby incorporated by reference in their entirety. In the event that one or more of the incorporated literature and similar materials differ from or contradict this application, including but not limited to defined terms, term usage, described techniques, or the like, this application controls.

What is claimed is:

1. A pharmaceutical composition comprising a therapeutically effective amount of a compound having the structure: 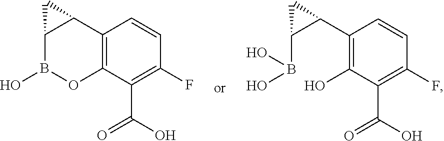 [SMILES: O=C(O)c1c(F)ccc2c1OB(O)[C@@H]1C[C@H]21 ; O=C(O)c1c(F)ccc([C@H]2C[C@H]2B(O)O)c1O]
or a pharmaceutically acceptable salt thereof, and a pharmaceutically acceptable excipient; and further comprising an additional medicament or a pharmaceutically acceptable salt thereof, wherein the additional medicament is cefiderocol, ceftolozane or sulbactam.

2. The pharmaceutical composition of claim 1, wherein the additional medicament is cefiderocol or a pharmaceutically acceptable salt thereof.

3. The pharmaceutical composition of claim 1, wherein the pharmaceutically acceptable salt is an alkaline metal salt or an ammonium salt.

4. The pharmaceutical composition of claim 3, wherein the pharmaceutically acceptable salt is a sodium salt.

5. The pharmaceutical composition claim 1, wherein the sodium salt is

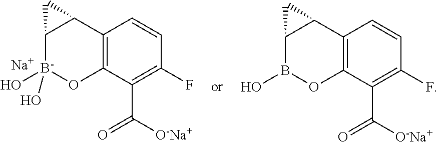

6. The pharmaceutical composition claim 1, wherein the sodium salt is

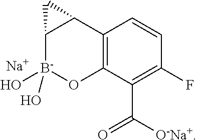

7. The pharmaceutical composition claim 1, wherein the sodium salt is 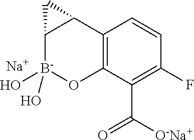
and wherein the additional medicament is cefiderocol or a pharmaceutically acceptable salt thereof.

8. The pharmaceutical composition of claim 1, wherein the sodium salt is [chemical structure]
